(19) United States
(12) Patent Application Publication
(10) Pub. No.: US 2024/0299418 A1
(43) Pub. Date: Sep. 12, 2024

(54) METHODS OF TREATING NEUROPATHIC PAIN

(71) Applicant: Robert Pelletier, Dixfield, ME (US)

(72) Inventor: Robert Pelletier, Dixfield, ME (US)

(21) Appl. No.: 18/599,626

(22) Filed: Mar. 8, 2024

Related U.S. Application Data
(60) Provisional application No. 63/489,251, filed on Mar. 9, 2023.

(51) Int. Cl. A61K 31/618; A61K 9/70; A61K 31/045; A61K 31/125; A61P 25/02

(52) U.S. Cl. CPC A61K 31/618; A61K 9/7084; A61K 31/045; A61K 31/125; A61P 25/02

(57) ABSTRACT

Disclosed are methods of treatment of neuropathic pain such as fibromyalgia induced neuropathic pain by use of patches, simultaneously, on the top and the bottom of a foot of an individual where the patches contain a formulation topically applied to the skin of the foot.

### CROSS REFERENCE TO RELATED APPLICATIONS

This application claims the benefit of and priority to U.S. Patent Application No. 63/489,251, filed on Mar. 9, 2023, the entire contents of which are incorporated by reference herein.

### BACKGROUND

Fibromyalgia is a chronic, debilitating disorder typified by widespread musculoskeletal pain, accompanied by symptoms of fatigue, affected sleep, memory issues, and mood disorders. It is estimated that fibromyalgia affects 2-8% of the American adult population. Women are more commonly diagnosed than men (female to male ratio is 2:1 based on newer diagnostic criteria) and prevalence increases with age. Fibromyalgia is considered by some researchers to be a condition that is largely mediated in the central nervous system given that fibromyalgia sufferers often present without a direct peripheral insult or injury. Since the 2011 revision of the IASP definition of neuropathic pain, fibromyalgia has been excluded from the diagnosis of neuropathic pain. More recent studies however found newer evidence of pathophysiology, including small fiber neuropathy in patients with fibromyalgia. This may challenge the existing consensus and have implications on future diagnosis and management of this condition. A recent expert review described fibromyalgia as the prototypical central chronic pain syndrome.

Individuals suffering from fibromyalgia presently have limited treatment options available. Current treatment options include pregabalin, a calcium channel alpha-2-delta subunit ligand, and two norepinephrine and serotonin re-uptake inhibitors, duloxetine and milnacipran. These therapies have shown limited efficacy in treating fibromyalgia symptoms., However, for a large proportion of patients, treatment is insufficient.

A need continues to exist in the art for novel and more specific and/or potent methods of treatment that are capable of delivering formulations for repressing or ceasing painful conditions such as neuropathic pain, for example, fibromyalgia. In addition, a need continues to exist in the medical arts for topically deliverable forms of such formulations.

### SUMMARY

The present disclosure includes methods of treating neuropathic pain in a patient in need thereof, comprising administering a first patch to the top portion of a foot of the patient and a second patch to the bottom of the foot of the patient, wherein each of a skin contacting surface of the first patch and of the second patch comprises a therapeutically effective amount of a formulation comprising menthol, camphor, and methyl salicylate thereby to topically deliver a therapeutically active amount of the formulation to treat the neuropathic pain. The first patch and the second patch can be adhered to the skin of the top and the bottom of the foot of the individual. The first patch and the second patch typically are simultaneously in place.

In some embodiments, the methods including treating a neuropathic pain or condition such as peripheral neuropathy, fibromyalgia, acute neuropathic pain, and chronic neuropathic pain, in a patient in need thereof. Such methods, as described herein, can include topically administering to the patient a therapeutically effective amount of a formulation as described herein.

### DESCRIPTION OF DRAWINGS

It should be understood that the drawings described below are for illustration purposes only. The drawings are not necessarily to scale, with emphasis generally being placed upon illustrating the principles of the present teachings. The drawings are not intended to limit the scope of the present teachings in any way.

FIG. 1 is a schematic diagram of an embodiment of the present teachings depicting two patches, where a first patch is positioned in contact with the top portion of a foot of the human and a second patch is positioned in contact with the bottom portion of the foot of the human.

FIG. 2 is a photograph of an embodiment of the present teachings showing the placement of embodiments of a first patch and a second patch on the foot of a human.

### DETAILED DESCRIPTION

It has now been discovered that the strategic placement of a patch on the top of an individual's foot and another patch on the bottom of the foot can be useful in the treatment of a variety of neuropathic pain related conditions such as fibromyalgia induced pain. More specifically, the present teachings provide methods of treating neuropathic pain in a patient in need thereof, comprising contacting a first patch with the top portion of a foot of the patient and contacting a second patch with the bottom of the foot of the patient, wherein a skin contacting surface of the first patch and of the second patch comprises a therapeutically effective amount of a formulation comprising menthol, camphor, and methyl salicylate thereby to treat the neuropathic pain. In various embodiments, the first patch and the second patch are simultaneously present on the foot.

Without wishing to be bound to any particular theory, it is believed that the foot has blood vessels and nerve endings closer to the surface of the skin than the rest of the body, and the feet act as a sort of a nerve gateway. So placing the patches on the feet in this way, top and bottom, tends to block most, if not all, of the pain signals traveling through the feet, including, for most or all of the body, or at least the lower extremities, which is where pain often is experienced for patients suffering with fibromyalgia. That is, the specific placement of the two patches can block the pain traveling through the feet to the brain, and very often can stop the pain within five minutes in the whole body.

The phrases “pharmaceutically acceptable” and “pharmacologically acceptable,” as used herein, refer to compounds, molecular entities, compositions, materials, and/or dosage forms that do not produce an adverse, allergic or other untoward reaction when administered to an animal, or a human, as appropriate.

The terms “individual,” “patient,” and “subject,” as used herein, are used interchangeably and include any animal, including mammals, preferably mice, rats, other rodents, rabbits, dogs, cats, swine, cattle, sheep, horses, or primates, and more preferably, humans. The compounds described in the disclosure can be administered to a mammal, such as a human, but can also be administered to other mammals such as an animal in need of veterinary treatment, for example, domestic animals (e.g., dogs, cats, and the like), farm animals (e.g., cows, sheep, pigs, horses, and the like) and laboratory animals (e.g., rats, mice, guinea pigs, and the like). The mammal treated in the methods described in the disclosure is preferably a mammal in which treatment, for example, of pain or depression, is desired.

The term “treating,” as used herein, includes any effect, for example, lessening, reducing, modulating, ameliorating, or eliminating, that results in the improvement of the condition, disease, disorder, and the like, including one or more symptoms thereof. Treating can be curing, improving, or at least partially ameliorating the disorder.

The phrase “therapeutically effective amount,” as used herein, refers to the amount of a compound or formulation (e.g., a disclosed compound or disclosed formulation, respectively) that will elicit the biological or medical response of a tissue, system, animal or human that is being sought by the researcher, veterinarian, medical doctor or other clinician. The compounds and formulations described in the disclosure can be administered in therapeutically effective amounts to treat a disease. A therapeutically effective amount of a compound or formulation can be the quantity required to achieve a desired therapeutic and/or prophylactic effect, such as an amount which results in lessening of a symptom of neuropathic pain.

Unless defined otherwise, all technical and scientific terms used herein have the same meaning as commonly understood by one of ordinary skill in the art to which this disclosure pertains.

Throughout the description, where compositions and kits are described as having, including, or comprising specific components, or where processes and methods are described as having, including, or comprising specific steps, it is contemplated that, additionally, there are compositions and kits of the present disclosure that consist essentially of, or consist of, the recited components, and that there are processes and methods according to the present disclosure that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present disclosure, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments can be variously combined or separated without parting from the present teachings and disclosure(s). For example, it will be appreciated that all features described and depicted herein can be applicable to all aspects of the disclosure(s) described and depicted herein.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present disclosure also include the specific quantitative value itself, unless specifically stated otherwise. As used herein, the term “about” refers to a ±10%, ±5%, ±3%, ±2%, or ±1% variation from the nominal value unless otherwise indicated or inferred but as understood as appropriate for the quantitative value and its measurement.

Where a percentage is provided with respect to an amount of a component or material in a composition, the percentage should be understood to be a percentage based on weight, unless otherwise stated or understood from the context.

Where a molecular weight is provided and not an absolute value, for example, of a polymer, then the molecular weight should be understood to be an average molecule weight, unless otherwise stated or understood from the context.

It should be understood that the order of steps or order for performing certain actions is immaterial so long as the present disclosure remain operable. Moreover, two or more steps or actions can be conducted simultaneously.

At various places in the present specification, substituents are disclosed in groups or in ranges. It is specifically intended that the description include each and every individual subcombination of the members of such groups and ranges. By way of example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present disclosure and does not pose a limitation on the scope of the disclosure unless claimed. No language in the specification should be construed as indicating any non-claimed element as essential to the practice of the present disclosure.

Various aspects of the disclosure are set forth herein under headings and/or in sections for clarity; however, it is understood that all aspects, embodiments, or features of the disclosure described in one particular section are not to be limited to that particular section but rather can apply to any aspect, embodiment, or feature of the present disclosure.

Described herein are methods of treating neuropathic pain in a patient in need thereof, comprising contacting a first patch with the top portion of a foot of the patient and contacting a second patch with the bottom of the foot of the patient, wherein a skin contacting surface of the first patch and of the second patch comprises a therapeutically effective amount of a formulation comprising menthol, camphor, and methyl salicylate thereby to treat the neuropathic pain.

In various embodiments, the formulation comprises capsaicin.

In various embodiments, the formulation comprises menthol in an amount between about 2% to about 15% by weight of the total weight of the formulation. In some embodiments, the formulation comprises menthol in an amount of between about 5% to about 9% by weight of the total weight of the formulation.

In various embodiments, the formulation comprises camphor in an amount between about 0.5% to 15% by weight of the total weight of the formulation. In some embodiments, the formulation comprises camphor in an amount between about 1% to 10% by weight of the total weight of the formulation.

In various embodiments, the formulation comprises methyl salicylate in an amount between about 1% to 15% by weight of the total weight of the formulation. In some embodiments, the formulation comprises methyl salicylate in an amount between about 5% to 10% by weight of the total weight of the formulation.

In various embodiments, the formulation comprises capsaicin in an amount between about 0.005% to 0.15% by weight of the total weight of the formulation. In some embodiments, the formulation comprises capsicum in an amount about 0.05% by weight of the total weight of the formulation. In certain embodiments, the formulation comprises capsicum extract in an amount about 3% by weight of the total weight of the formulation.

In various embodiments, the formulation further comprises one or more of a saturated hydrocarbon resin, lanolin, natural rubber, styrene-isoprene-styrene block co-polymer, petrolatum, polyisobutylene, rosin, zinc oxide, paraffin, aluminum silicate, and peppermint oil.

In various embodiments, the first patch and the second patch are rectangular in shape. In some embodiments, the skin contacting surface of the first patch and of the second patch has an area of between about 20 cm to about 75 cm. In certain embodiments, the skin contacting surface of the first patch and of the second patch has an area of between about 30 cm to about 55 cm.

In various embodiments, each of the first patch and the second patch comprises an adhesive portion along opposing sides of the first patch and along opposing sides of the second patch, whereby the first patch and the second patch are kept in contact with the top of the foot and the bottom of the foot, respectively, by the adhesive portions.

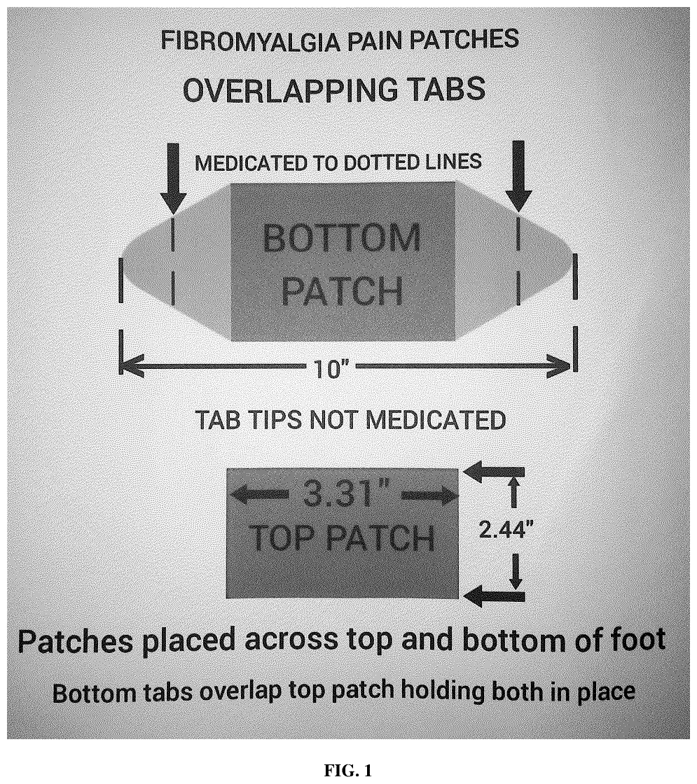

FIG. 1 is a schematic diagram of an embodiment of the present teachings depicting two patches, where a first patch is positioned in contact with the top portion of a foot of the human and a second patch is positioned in contact with the bottom portion of the foot of the human. In this embodiment, the bottom patch has tabs projecting from opposite sides, where a portion of the tab is medicated and the other portion can contain an adhesive such that when in use, the bottom tabs overlap the top patch, holding both patches in place. Of course the dimensions shown in FIG. 1 are only illustrative as the patch sizes can be adjusted for a particular human's foot size.

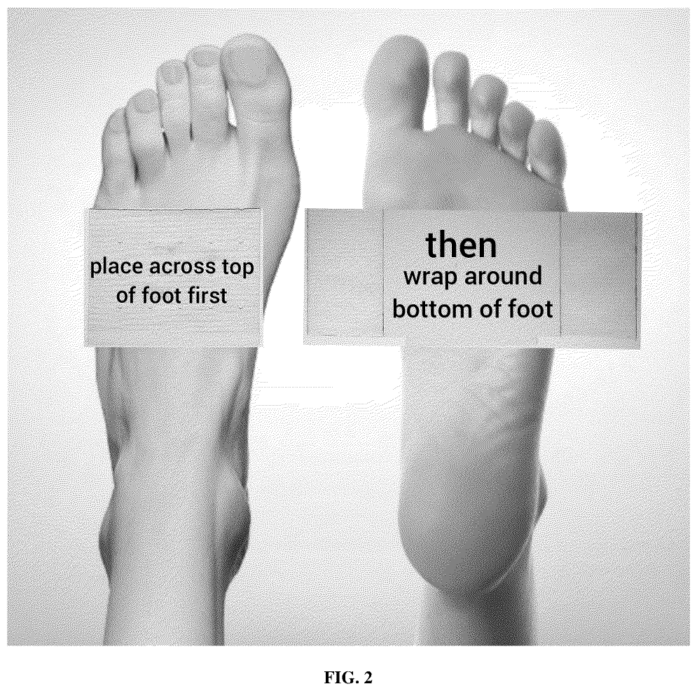

FIG. 2 is a photograph showing the placement of embodiments of a first patch and a second patch on the foot of a human for the treatment of neuropathic pain such as fibromyalgia.

In some embodiments, methods are provided for treating neuropathic pain. The neuropathic pain can be acute or chronic. In some cases, the neuropathic pain can be associated with a condition such as chronic back pain induced, fibromyalgia induced, and diabetic neuropathy induced such as diabetic peripheral neuropathy induced neuropathic pain. In certain embodiments, methods for enhancing pain relief and for providing analgesia to a patient are also provided.

### EXAMPLES

The following examples are provided for illustrative purposes only, and are not intended to limit the scope of the disclosure.

Although the formulations and patches can be made as described herein, commercially available patches fall within the present teachings, as described below. Thus, it should be understood that the invention lies in the placement of the patches, which is what provides the treatment and/or prevention of neuropathic pain such as fibromyalgia as described herein.

### Example 1

A commercially available patch, Coralite® Muscle & Joint Extra Strength Pain Relief Patch, can be used in practice of the present teachings. The formulation on the skin contacting surface of the patch contains about 1.2% camphor, 5.7% menthol, and 6.3% methyl salicylate. One 12.4 cm by 8.4 cm patch was cut in half, with an adhesive portion positioned along opposing sides of each patch so that it can be held against the skin of a foot.

### Example 2

Another commercially available patch, Sumipad®, Pain Relief Patch (7 cm by 10 cm) was cut in half in the same fashion as with the patch in Example 1, with adhesive portions added thereto in the same fashion. The formulation on the skin contacting surface of the patch contains about 2.89% capsicum extract, 7.8% menthol, and 9.6% camphor. Because of the capsaicin and extra camphor compared to the patched in Example 1, this patch is considered to be of a higher strength than the patches in Example 1.

### Example 3

An individual was experiencing pain caused by fibromyalgia. The patches made according to Example 1 were applied to the individual, one patch was adhered to the top part of the individual's foot and another patch was adhered to the bottom of the same foot. Within 5 minutes, the pain began to wane and after 30 minutes the individual was nearly pain-free and remained that way for over 12 hours.

### Example 4

An individual was experiencing severe pain caused by fibromyalgia. The patches made according to Example 2 were applied to the individual, one patch was adhered to the top part of the individual's foot and another patch was adhered to the bottom of the same foot. Within 10 minutes, the pain began to wane and after about one hour the individual was nearly pain-free and remained that way for over 18 hours.

### EQUIVALENTS

The disclosure may be embodied in other specific forms without departing from the spirit or essential characteristics thereof. The foregoing embodiments are therefore to be considered in all respects illustrative rather than limiting the disclosure described herein. Those skilled in the art will recognize, or be able to ascertain using no more than routine experimentation, many equivalents to the specific embodiments of the disclosure described herein. Such equivalents are intended to be encompassed by the following claims. Scope of the invention is thus indicated by the appended claims rather than by the foregoing description, and all changes that come within the meaning and range of equivalency of the claims are intended to be embraced therein.

### INCORPORATION BY REFERENCE

The entire contents of all patents, published patent applications, websites, and other references cited herein are incorporated by reference herein in their entireties.

1. A method of treating neuropathic pain in a human in need thereof, comprising contacting a first patch with the top portion of a foot of the human and contacting a second patch with the bottom of the foot of the human, wherein a skin contacting surface of the first patch and of the second patch comprises a therapeutically effective amount of a formulation comprising menthol, camphor, and methyl salicylate thereby to treat the neuropathic pain.

2. A method of treating neuropathic pain of claim 1, wherein the formulation comprises menthol in an amount between about 2% to about 15% by weight of the total weight of the formulation.

3. The method treating neuropathic pain of claim 1, wherein the formulation comprises menthol in an amount of between about 5% to about 9% by weight of the total weight of the formulation

4. The method treating neuropathic pain of claim 1, wherein the formulation comprises camphor in an amount between about 0.5% to 15% by weight of the total weight of the formulation.

5. The method treating neuropathic pain of claim 2, wherein the formulation comprises camphor in an amount between about 0.5% to 15% by weight of the total weight of the formulation.

6. The method treating neuropathic pain of claim 1, wherein the formulation comprises camphor in an amount between about 1% to 10% by weight of the total weight of the formulation.

7. The method treating neuropathic pain of claim 1, wherein the formulation comprises methyl salicylate in an amount between about 1% to 15% by weight of the total weight of the formulation.

8. The method treating neuropathic pain of claim 2, wherein the formulation comprises methyl salicylate in an amount between about 1% to 15% by weight of the total weight of the formulation.

9. The method treating neuropathic pain of claim 5, wherein the formulation comprises methyl salicylate in an amount between about 1% to 15% by weight of the total weight of the formulation.

10. The method treating neuropathic pain of claim 1, wherein the formulation comprises methyl salicylate in an amount between about 5% to 10% by weight of the total weight of the formulation.

11. The method treating neuropathic pain of claim 1, wherein the formulation further comprises one or more of a saturated hydrocarbon resin, lanolin, natural rubber, styrene-isoprene-styrene block co-polymer, petrolatum, polyisobutylene, rosin, zinc oxide, paraffin, aluminum silicate, and peppermint oil.

12. The method treating neuropathic pain of claim 1, wherein the first patch and the second patch are rectangular in shape.

13. The method treating neuropathic pain of claim 1, wherein the skin contacting surface of the first patch and of the second patch has an area of between about 20 cm to about 75 cm.

14. The method treating neuropathic pain of claim 1, wherein the skin contacting surface of the first patch and of the second patch has an area of between about 30 cm to about 55 cm.

15. The method treating neuropathic pain of claim 1, wherein the neuropathic pain is chronic or acute.

16. The method treating neuropathic pain of claim 1, wherein the neuropathic pain is fibromyalgia induced neuropathic pain.

17. The method treating neuropathic pain of claim 1, wherein the first patch and the second patch are simultaneously present on the foot.

18. The method treating neuropathic pain of claim 1, wherein each of the first patch and the second patch comprises an adhesive portion along opposing sides of the first patch and along opposing sides of the second patch, whereby the first patch and the second patch are kept in contact with the top of the foot and the bottom of the foot, respectively, by the adhesive portions.

19. The method treating neuropathic pain of claim 1, wherein one of the first patch and the second patch comprises an adhesive portion along opposing sides, whereby the first patch and the second patch are kept in contact with the top of the foot and the bottom of the foot, respectively, by the adhesive portion that wraps around the foot to contact the other of the first patch and the second patch that does not comprise an adhesive.
